(19) United States
(12) Patent Application Publication
(10) Pub. No.: US 2021/0406025 A1
(43) Pub. Date: Dec. 30, 2021

(54) METHOD OF AND SYSTEM FOR GENERATING A RANK-ORDERED INSTRUCTION SET USING A RANKING PROCESS

(71) Applicant: KPN INNOVATIONS, LLC, LAKEWOOD, CO (US)

(72) Inventor: Kenneth Neumann, Lakewood, CO (US)

(21) Appl. No.: 16/912,089

(22) Filed: Jun. 25, 2020

(51) Int. Cl. G06F 9/38; G06N 20/00

(52) U.S. Cl. CPC G06F 9/3851; G06N 20/00

(57) ABSTRACT

A system for generating rank-ordered instruction sets includes at least a computing device, wherein the at least a computing device is configured to generate a first rank-ordered list of instructions, wherein generating further comprises receiving a plurality of user objectives, determine, using a first ranking process and a plurality of objectives, a rank-ordered objective set, identify, using a first machine-learning process and ranked-ordered goal set, an instruction set including a plurality of instructions, wherein the plurality of instructions includes an instruction for addressing each objective of the plurality of objectives, generate, using a second ranking process and a first plurality of instructions, the first ranked-ordered list of instructions for addressing the rank-ordered objective set. provide the rank-ordered instruction set to a user device, receive, from the user device, a plurality of user data, and generate, using the plurality of user data, a second rank-ordered list of instructions.

### FIELD OF THE INVENTION

The present invention generally relates to the field of machine-learning. In particular, the present invention is directed to a method of and system for generating a rank-ordered instruction set using a ranking process.

### BACKGROUND

Machine-learning methods are increasingly valuable for analysis of patterns in large quantities of data. However, where the data is large and varied enough, optimizing instructions for users from machine-learning outputs can become untenable, especially with tradeoffs between sophistication and efficiency.

### SUMMARY OF THE DISCLOSURE

In an aspect a system for generating a rank-ordered instruction set using a ranking process, the system comprising at least a computing device, wherein the at least a computing device is configured to generate a first rank-ordered list of instructions, wherein generating further comprises receiving a plurality of user objectives, determining, using a first ranking process and a plurality of objectives, a rank-ordered objective set, identifying, using a first machine-learning process and ranked-ordered objective set, an instruction set including a plurality of instructions, wherein the plurality of instructions includes an instruction for addressing each objective of the plurality of objectives, and generating, using a second ranking process and a first plurality of instructions, the first ranked-ordered list of instructions for addressing the rank-ordered objective set. Computing device is configured to provide the rank-ordered objective set to a user device. Computing device receives from the user device, a plurality of user data. Computing device generates, using the plurality of user data, a second rank-ordered list of instructions.

In another aspect a method for generating a rank-ordered instruction set using a ranking process, the system comprising at least a computing device, wherein the at least a computing device is configured to generate a first rank-ordered list of instructions, wherein generating further comprises receiving a plurality of user objectives, determining, using a first ranking process and a plurality of objectives, a rank-ordered objective set, identifying, using a first machine-learning process and ranked-ordered objective set, an instruction set including a plurality of instructions, wherein the plurality of instructions includes an instruction for addressing each objective of the plurality of objectives, and generating, using a second ranking process and a first plurality of instructions, the first ranked-ordered list of instructions for addressing the rank-ordered objective set. Computing device is configured to provide the rank-ordered objective set to a user device. Computing device receives from the user device, a plurality of user data. Computing device generates, using the plurality of user data, a second rank-ordered list of instructions.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for generating a rank-ordered instruction set using objective functions;

FIG. 2 is a block diagram illustrating an exemplary embodiment of a user database;

FIG. 3 is a diagrammatic representation of a plurality of objectives prior to applying instructions;

FIG. 4 is a diagrammatic representation of a plurality of objectives after applying proposed instruction sets;

FIG. 5 is a diagrammatic representation of an exemplary embodiment of a user device for receiving rank-ordered objective set and rank-ordered instruction set;

FIG. 6 is a flow diagram illustrating a method of generating rank-ordered instruction sets using a ranking process; and

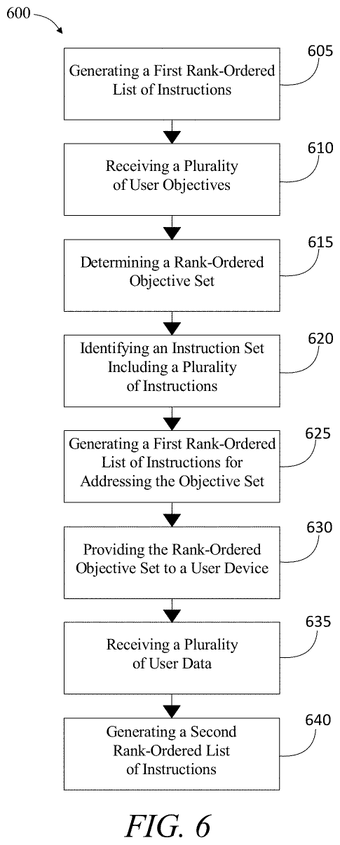

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, embodiments described herein improve speed and accuracy in generating rank-ordered instruction sets for users to achieve a set of objectives by selecting a subset of maximally impactful solutions and ranking instructions in a meaningful order for a user to follow. Objective functions may be used to rank the subset based on numerical ranking derived from a machine-learning process. Further classification of biological extraction data to objectives may enable detection and alleviation thereof in users. Machine-learning process may iteratively improve subsets of solutions by calculating impact of user action in response to instruction sets.

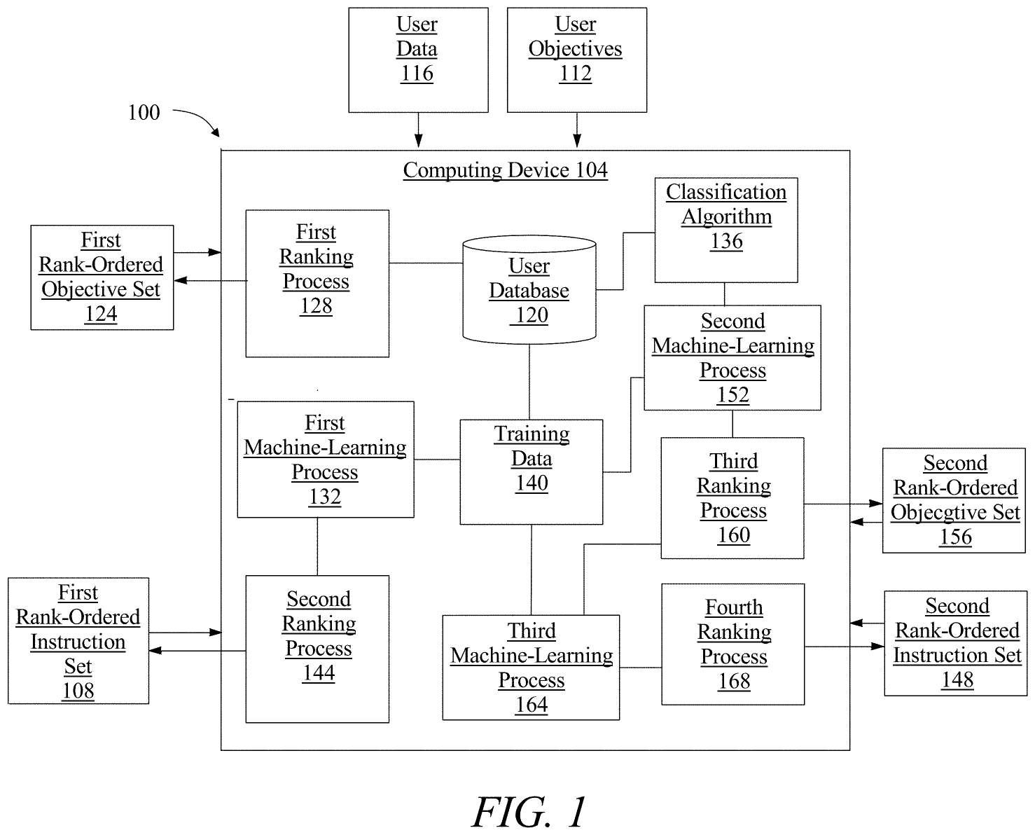

Referring now to FIG. 1, an exemplary embodiment of a system 100 for generating rank-ordered instruction sets using a ranking process is illustrated. System 100 includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. computing device 104 may include but is not limited to, for example, a computing device or cluster of computing devices in a first location and a second computing device or cluster of computing devices in a second location. computing device 104 may include one or more computing devices dedicated to data storage, security, distribution of traffic for load balancing, and the like. computing device 104 may distribute one or more computing tasks as described below across a plurality of computing devices of computing device, which may operate in parallel, in series, redundantly, or in any other manner used for distribution of tasks or memory between computing devices. computing device 104 may be implemented using a “shared nothing” architecture in which data is cached at the worker, in an embodiment, this may enable scalability of system 100 and/or computing device.

Computing device 104 may be configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 is configured to generate a first rank-ordered instruction set 108. Generating may include receiving a plurality of user objectives. Receiving a plurality of user objectives 112 may include receiving at least a user-reported objective. A “user-reported objective,” as used in this disclosure is an objective directly input by a user; for instance and without limitation, a user may input an objective of reducing body fat, an objective to quit smoking, an objective to improve mental plasticity, or the like. Receiving at least an objective may include objectives that are determined from a plurality of data associated with user, such as user-reported data, other user data, and/or data reported by another person and/or device, for instance and without limitation, by a machine-learning process analyzing user data 116 and/or supplied by a physician from medical history data. User data 116 as used herein may include, for instance, data used as a biological extraction as described in U.S. Nonprovisional application Ser. No. 16/502,835, filed on Jul. 3, 2019, and entitled “METHODS AND SYSTEMS FOR ACHIEVING VIBRANT CONSTITUTION BASED ON USER INPUTS,” the entirety of which is incorporated herein by reference. User objectives 112 may include objectives specific to a user that may be received by a computing device 104 from multiple sources. In non-limiting examples, user objectives 112 may be retrieved, without limitation, from a user database 120 by a computing device 104 as described in further detail below, user objectives 112 may be input by personnel other than a first user, for instance from a physician, laboratory technician, nurse, caregiver, or the like, via for instance, a telemedicine platform. User objectives 112 may be stored and/or retrieved from a database, server, or the like for subsequent ranking process inputs, machine-learning process inputs, or the like, as described in further detail below. Persons skilled in the art, upon reviewing the entirety of this disclosure, will be aware of various ways in which objectives and/or sequences of objectives, may be input and/or collected by a computing device 104.

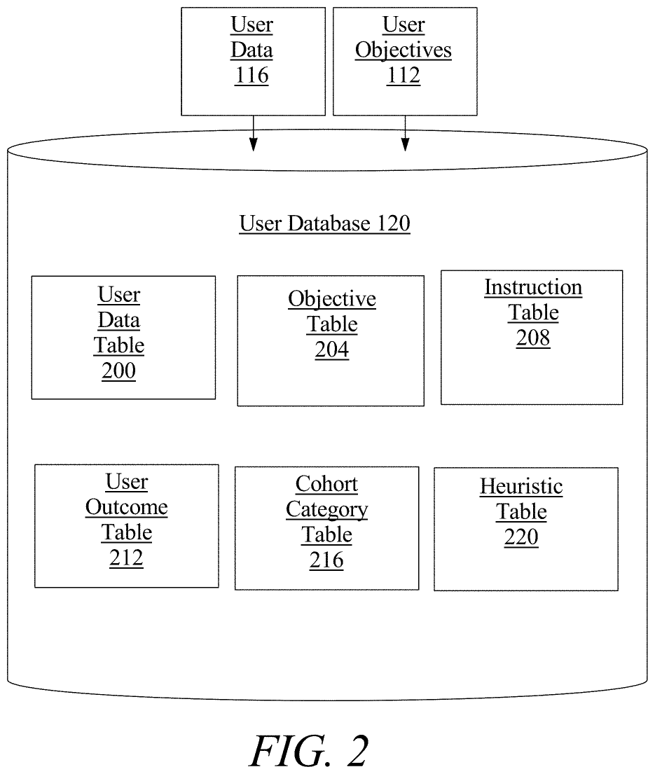

Referring now to FIG. 2, a non-limiting exemplary embodiment of a user database 120 is illustrated. Database may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

Database may refer to a “user database” which at least a computing device 104 may, alternatively or additionally, store and/or retrieve data from a user data table 200, goal table, 204 and/or instruction table 208. Determinations by a machine-learning process may also be stored and/or retrieved from the user database 120, for instance in non-limiting examples a classifier describing a subset of data. As a non-limiting example, user database 120 may organize data according to one or more instruction tables. One or more user database 120 tables may be linked to one another by, for instance in a non-limiting example, common column values. For instance, a common column between two tables of user database 120 may include an identifier of a submission, such as a form entry, textual submission, research paper, or the like, for instance as defined below; as a result, a query may be able to retrieve all rows from any table pertaining to a given submission or set thereof. Other columns may include any other category usable for organization or subdivision of expert data, including types of expert data, names and/or identifiers of experts submitting the data, times of submission, or the like; persons skilled in the art, upon reviewing the entirety of this disclosure, will be aware of various ways in which data from one or more tables may be linked and/or related to data in one or more other tables.

Still referring to FIG. 2, in a non-limiting embodiment, one or more tables of a user database 120 may include, as a non-limiting example, a user data table 200, which may include biological extraction analyses for use in predicting objectives of a user and/or instructions for a user and/or correlating user data to other users, entries indicating degrees of relevance to and/or efficacy in predicting an objective of a user, and/or other elements of data computing device 104 and/or system 100 may use to determine usefulness and/or relevance of user data in determining objectives, instructions, and/or changes in objectives and/or instructions as described in this disclosure. One or more tables may include an objective table 204, which may include a history of objectives corresponding to a user, for instance and without limitation, that a user has held, obtained, still left to attain, and other identifying information linked to the attainment of objectives, for instance the number, type, and efficacy of instructions in achieving an objective, length of time to achieve an objective, and an objective's associated tractability, among other information. One or more tables may include an instruction table 208, which may correlate user data, objectives, outcomes, models, heuristics, and/or combinations thereof to one or more measures of achieving an objective; One or more tables may include, without limitation, a user outcome table 212 which may contain one or more inputs identifying one or more categories of data, for instance numerical values describing the propensity of a user to follow an instruction, or the long-term effect an instruction has on future objectives, and the like. One or more tables may include, without limitation, a cohort category table 216 which may contain one or more inputs identifying one or more categories of data, for instance demographic data, physiological data, sleep pattern data, spending data, or the like, with regard to which users having matching or similar data may be expected to have similar objectives and/or instruction sets as a result of ranking process output elements and/or other user data input elements. One or more tables may include, without limitation, a heuristic table 220, which may include one or more inputs describing potential mathematical relationships between at least an element of user data and objectives, instructions, and rankings thereof, change in objectives and/or instructions over time, and/or ranking functions for determining a rank-ordered set of objectives and/or instructions, as described in further detail below.

Referring now to FIG. 1, a computing device 104 may be configured to generate a first rank-ordered instruction set 108 which may include using a first ranking process and a first plurality of user objectives to determine a first rank-ordered goal set 124. A “ranking process,” as described herein refers to ranking performed by any ‘objective function’ used by a computing device 104 to place elements in an optimal listing based upon a score, measure, or numerical value, as described in further detail below. A computing device 104 may compute a score associated with each goal and select objectives to minimize and/or maximize the score, depending on whether an optimal result is represented, respectively, by a minimal and/or maximal score; a mathematical function, described herein as an “objective function,” may be used by computing device 104 to score each possible pairing. Objective function may be based on one or more objectives, as described below. Computing device 104 may pair a predicted route, with a given courier, that optimizes objective function. In various embodiments a score of a particular goal may be based on a combination of one or more factors, including user data 116. Each factor may be assigned a score based on predetermined variables. In some embodiments, the assigned scores may be weighted or unweighted, for instance and without limitation as described in the U.S. Nonprovisional application Ser. No. 16/890,686, filed on Jun. 2, 2020, and entitled “ARTIFICIAL INTELLIGENCE METHODS AND SYSTEMS FOR CONSTITUTIONAL ANALYSIS USING OBJECTIVE FUNCTIONS,” the entirety of which is incorporated herein by reference.

Optimization of an objective function may include performing a greedy algorithm process. A “greedy algorithm” is defined as an algorithm that selects locally optimal choices, which may or may not generate a globally optimal solution. For instance, computing device 104 may select objectives so that scores associated therewith are the best score for each goal. For instance, in non-limiting illustrative example, optimization may determine the combination of routes for a courier such that each delivery pairing includes the highest score possible, and thus the most optimal delivery.

Still referring to FIG. 1, objective function may be formulated as a linear objective function, which computing device 104 may solve using a linear program such as without limitation a mixed-integer program. A “linear program,” as used in this disclosure, is a program that optimizes a linear objective function, given at least a constraint; a linear program maybe referred to without limitation as a “linear optimization” process and/or algorithm. For instance, in non-limiting illustrative examples, a given constraint might be a nutritional deficiency of a user, and a linear program may use a linear objective function to calculate minimized caloric intake for weight loss without exacerbating a nutritional deficiency. In various embodiments, system 100 may determine a set of instructions towards achieving a user's goal that maximizes a total score subject to a constraint that there are other competing objectives. A mathematical solver may be implemented to solve for the set of instructions that maximizes scores; mathematical solver may be implemented on computing device 104 and/or another device in system 100, and/or may be implemented on third-party solver.

With continued reference to FIG. 1, optimizing objective function may include minimizing a loss function, where a “loss function” is an expression an output of which a ranking process minimizes to generate an optimal result. As a non-limiting example, computing device 104 may assign variables relating to a set of parameters, which may correspond to score components as described above, calculate an output of mathematical expression using the variables, and select an objective that produces an output having the lowest size, according to a given definition of “size,” of the set of outputs representing each of plurality of candidate ingredient combinations; size may, for instance, included absolute value, numerical size, or the like. Selection of different loss functions may result in identification of different potential pairings as generating minimal outputs

Continuing in reference to FIG. 1, generating a first rank-ordered instruction set 108 may include determining, using a first ranking process and a plurality of objectives, a rank-ordered goal set. A ranking process may include any of the functions described above, such as a linear objective function that may input a plurality of user-reported objectives, and rank the objectives by a variety of factors, for instance without limitation, by impact to health, and output a first rank-ordered goal set 124 ranked by that function. Determining a first rank-ordered goal set 124 set may include using a first ranking process using a ranking function to determine the relative importance of an objective, for instance and without limitation in Table 1. In non-limiting illustrative examples, an objective function may include use of a ranking function to determine a rank-order for objectives based on a numerical value, index, matrix, or the like, to determine the goal rank order for a user.

Values generated by ranking process may include, as a non-limiting example, using values from a ranking function as illustrated in Table 1 below:

TABLE 1

| Score | Weight |
|---|---|
| x ≤ −2.5 | x(1.4) |
| −2.5 < x ≤ −1.5 | x(1.3) |
| −1.5 < x ≤ −1.0 | x(1.2) |
| −1.0 < x ≤ −0.5 | x(1.1) |
| −0.5 < x ≤ +0.0 | x(1.0) |
| +0.0 < x ≤ +0.5 | x(1.0) |
| +0.5 < x ≤ +1.0 | x(0.9) |
| +1.0 < x ≤ +1.5 | x(0.7) |
| +1.5 < x ≤ +2.5 | x(0.5) |
| +2.5 < x | x(0.3) |

In non-limiting illustrative examples ranking function may include a mathematical or other function that was retrieved from a user database 120, calculated by a machine-learning process, or otherwise obtained that provides qualitative and/or quantitative guidance in determining a rank for an objective. Ranking function may contain values derived from user data 116 to determine a priority listing for objectives based on, for instance without limitation severity of health concern.

Continuing in reference to FIG. 1, generating a first rank-ordered instruction set 108 may include identifying, using a first machine-learning process 132 and first ranked-ordered goal set 124, an instruction set including a plurality of instructions, wherein the plurality of instructions includes an instruction for addressing each goal of the plurality of objectives. A first machine-learning process 132 may include a machine-learning process. A machine-learning process may include at least a supervised machine-learning process. Supervised machine learning processes, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some ranking function. For instance, a supervised learning algorithm may include a plurality of objectives as described above as inputs, a plurality of instructions to address the objectives as outputs, and a ranking function representing a desired form of relationship to be detected between inputs and outputs; ranking function may, for instance, seek to maximize the probability that a given input and/or combination of elements inputs is associated with a given output to minimize the probability that a given input is not associated with a given output. Ranking function may be expressed as a risk function representing an “expected loss” of an algorithm relating inputs to outputs, where loss is computed as an error function representing a degree to which a prediction generated by the relation is incorrect when compared to a given input-output pair provided in training data. Persons skilled in the art, upon reviewing the entirety of this disclosure, will be aware of various possible variations of supervised machine learning algorithms that may be used to determine relation between inputs and outputs.

Supervised machine learning processes may include classification algorithms 136, defined as processes whereby at least a computing device 104 derives, from training data, a model for sorting inputs into categories or bins of data. Classification may be performed using, without limitation, linear classifiers such as without limitation logistic regression and/or naive Bayes classifiers, regression algorithms, nearest neighbor classifiers, support vector machines, decision trees, boosted trees, random forest classifiers, and/or neural network-based classifiers, such as supervised neural net algorithms. Supervised machine learning processes may include, without limitation, machine learning processes as described in U.S. Nonprovisional application Ser. No. 16/520,835, filed on Jul. 3, 2019, and entitled “METHODS AND SYSTEMS FOR ACHIEVING VIBRANT CONSTITUTION BASED ON USER INPUTS,” the entirety of which is incorporated herein by reference.

Continuing in reference to FIG. 1, “training data,” as used herein, is data containing correlations that a machine-learning process may use to model relationships between two or more categories of data elements. For instance, and without limitation, training data 140 may include a plurality of data entries, each entry representing a set of data elements that were recorded, received, and/or generated together; data elements may be correlated by shared existence in a given data entry, by proximity in a given data entry, or the like. Multiple data entries in training data 140 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 140 according to various correlations; correlations may indicate causative and/or predictive links between categories of data elements, which may be modeled as relationships such as mathematical relationships by machine learning processes as described in further detail below. Training data 140 may be formatted and/or organized by categories of data elements, for instance by associating data elements with one or more descriptors corresponding to categories of data elements. As a non-limiting example, training data 140 may include data entered in standardized forms by persons or processes, such that entry of a given data element in a given field in a form may be mapped to one or more descriptors of categories. Elements in training data 140 may be linked to descriptors of categories by tags, tokens, or other data elements; for instance, and without limitation, training data 140 may be provided in fixed-length formats, formats linking positions of data to categories such as comma-separated value (CSV) formats and/or self-describing formats such as extensible markup language (XML), enabling processes or devices to detect categories of data.

Alternatively or additionally, training data 140 may include one or more elements that are not categorized; that is, training data 140 may not be formatted or contain descriptors for some elements of data. Machine learning algorithms and/or other processes may sort training data 140 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine learning algorithms, and/or automated association of data in the data entry with descriptors or into a given format. The ability to categorize data entries automatedly may enable the same training data 140 to be made applicable for two or more distinct machine learning algorithms as described in further detail below. Training data 140 used by computing device 104 may correlate any input data as described in this disclosure to any output data as described in this disclosure. Training data may contain entries, each of which correlates a machine learning process input to a machine learning process output, for instance without limitation, one or more elements of biological extraction data to a taste index. Training data may be obtained from previous iterations of machine-learning processes, user inputs, and/or expert inputs.

Still referring to FIG. 1, computing device 104 may calculate at least a plurality of instructions for a user using a first machine-learning process 132 and at least an element of a rank-ordered goal set 124 to generate, as an output, at least a instruction for a user of a plurality of instructions. Computing device 104 may generate an instruction set by training a first machine-learning process 132 with training data 140 correlating user data 116 with a first rank-ordered goal set 124, and calculating at least a first instruction as a function of at least a first element of biological extraction data. As described herein, “instruction,” refers to at least a step, incremental change, intervention, or action of a user carried out with some effect on a plurality of objectives. A machine-learning process, and/or a machine-learning model produced thereby, may be trained by at least a computing device 104 using training data, which may be retrieved from a user database 120, as described above, as it correlates to user data 116. A machine-learning process may be trained by using training data, for instance and without limitation, blood test results as it relates to nicotine and a determined instruction set for weaning a user off cigarettes as it relates to instruction sets provided to other users with blood test results signaling similar levels of cigarette use. In further non-limiting illustrative examples, such an instruction set would be output by a machine-learning process that may use a model trained with training data relating instruction sets provided to other users that may have a varying degree of similarity in blood test results.

Continuing in reference to FIG. 1, computing device 104 may be configured to calculate at least a plurality of instructions by retrieving an instruction from a user database 120. In non-limiting illustrative embodiments, a first machine-learning process 132 may receive a first rank-ordered goal set 124 from a first ranking process 128, wherein objectives may be ranked using a ranking function relating user preference data, health data, lifestyle data, and the like. In non-limiting illustrative embodiments, machine-learning process may determine a course of action for a user to work towards achieving at least an objective by retrieving information, for instance from an online repository, user database 120, or the like. In further non-limiting embodiments, machine learning process retrieving information from a user database 120 may query, for instance and without limitation, solutions for achieving an objective related to quitting smoking, calculating the anticipated impact of each solution on the goal, and then filter solutions based on a variety of methods, as described in further detail below.

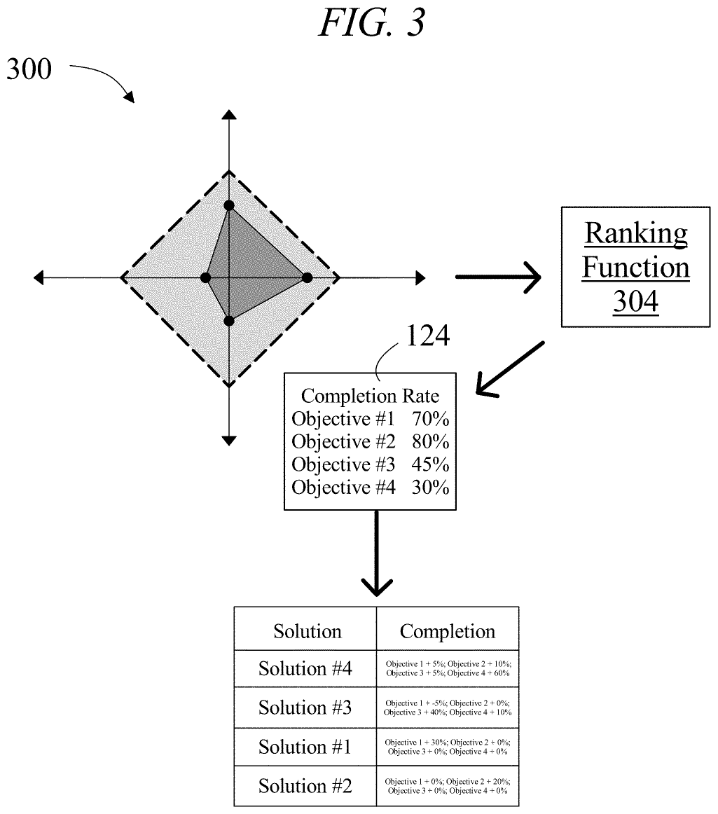

Referring now to FIG. 3, an exemplary embodiment of a first set of rank-ordered objectives 124 prior to applying solutions 300 is illustrated. Each goal of the plurality of rank-ordered objectives is rank-ordered by first ranking process 128 after applying a ranking function 304. A first rank-ordered goal set may be input into a first machine learning process 132 to identify and/or measure an effect of at least a solution towards achieving at least an objective. The first machine-learning process 132 may then tabulate all instructions sufficient to fulfill one or more requirements of an objective and output an instruction set necessary to achieve a set of objectives.

Referring now to FIG. 1, a computing device 104 configured to identify an instruction set to address an objective using a first machine-learning process 132 may include measuring an effect of a solution on an objective. A first machine-learning process 132 may measure the effect of an instruction, describing an incremental step of a solution, on an objective by factoring completion of an instruction towards achieving an objective. Measuring an effect of an instruction may include performing any mathematical function such as subtraction, applying a ranking function, matrix, system of equations, or any other calculation performed by a machine-learning process. In non-limiting illustrative embodiments, a first machine-learning process 132 may retrieve at least an element of data from a user database 120, such as a metric, indicator, or the like, that relates level of completion of an instruction towards achieving an objective. In non-limiting illustrative examples, a first machine-learning process 132 may query a database for solutions to an objective of ‘quitting smoking’ or ‘remove cigarette addiction’ and retrieve a table of values that correspond to a user's level of addiction to physiological levels of nicotine, for instance in the blood. In further non-limiting illustrative examples, a first machine-learning process 132 may employ a mathematical function to determine from biological extraction data such as blood chemistry data, among other forms of user data, the level of nicotine a user may obtain from his or her addiction; a first machine-learning process 132 may then determine a schedule of nicotine microdosing to wean a user off cigarettes with the end goal of eliminating nicotine intake. In non-limiting illustrative examples, a first machine-learning process may identify a solution of ‘take 4 mg nicotine every 2 hours, up to 20 mg daily’ to match current user nicotine use, with a tiered schedule in decreasing weekly dosages according to what was found to be efficacious. Although many illustrative examples provided herein apply to physiological objectives, processes and/or process steps disclosed herein may alternatively or additionally be applied to instructions to achieve other objectives. For instance and without limitation, a first machine-learning process 132 may measure completion of an instruction which may include paying off a bank loan, over a defined period of time, in working towards an objective of ‘improving a user's credit score’. In further non-limiting illustrative examples, a machine-learning process may retrieve a metric from a user database 120 that relates a dollar amount of debt payment, with a loan age, a period of time, and a current user credit score, to calculate how completion of the instruction impacts the user's overall credit score; the first machine-learning process 132 may then perform a mathematical function, such as subtraction, to determine if this change in credit score has improved the overall credit score enough for completion of the goal. In further non-limiting illustrative examples, a machine-learning process solution may be ‘paying off all user debts’ towards achieving an objective of ‘improving credit score’, wherein an instruction set of the solution may be dollar amounts toward individual loans, a time period for paying loans, and dates of submitting payments for varying levels of impact toward a credit score. In non-limiting illustrative examples, a first machine-learning process 132 may retrieve user data 116 from a user database 120 relating to implementing such an instruction set, for instance net income, gross income, secondary debt obligations, cost-of-living, and the like, in measuring an effect of a solution, and the subset of instructions for that solution, in achieving the goal.

Continuing in reference to FIG. 1, identifying an instruction set to address an objective using a first machine-learning process 132 may include eliminating, using the first machine learning process 132, redundant solutions in addressing at least an objective of a first rank-ordered goal set 124. Redundant solutions may be solutions that result in instruction sets that overlap, at least in part, with respect to how a user might perform the instructions. For instance and without limitation, a first machine-learning process 132 may eliminate solutions that may be redundant in addressing at least an objective, such as retrieving a plurality of solutions after querying ‘how to improve sleep quality’ and/or ‘establishing improved circadian rhythm cycles’, and returning ‘reduce interaction with electronic devices within 2 hours of sleep’ and ‘reduce mobile phone usage within 2 hours of sleep’. The first machine-learning process 132 may combine the two instructions into one or eliminate one of the instructions, as they overlap. In continued non-limiting illustrative examples, the first machine-learning process 132 may calculate or otherwise determine the magnitude of effect of ‘reducing interaction with electronic devices and/or mobile phone within 2 hour of sleep’ has on improving sleep quality and establishing circadian rhythm for a user, and this may results in for instance a sustained effort of following this instruction for at least 4 weeks before having an effect on circadian rhythm and sleep quality, or 2 weeks if combined with a second instruction of ‘take a warm bath 1 hour prior to sleep’ and/or a third instruction of ‘take 10 mg of melatonin 1 hour prior to sleep’. In further non-limiting illustrative examples, first machine-learning process 132 may output all three instructions with information indicating that one of the instructions be eliminated in favor of another, for instance at a certain point in time, or because more than one instruction may not be necessary.

Continuing in reference to FIG. 1, identifying an instruction set to address an objective may include reconciling, using first machine learning process 132, opposing solutions in addressing at least an objective. For instance and without limitation, first machine-learning process 132 may reconcile solutions that may be opposing in practice in addressing at least an objective, such as retrieving a plurality of solutions after querying ‘how to improve body composition’ and/or ‘losing body fat and gaining muscle’, and returning ‘reduce caloric intake to 1,800 calories’ and ‘increase protein intake to 2 grams protein per kilogram body weight’. In continuing non-limiting illustrative examples, a machine-learning process may retrieve from a user database 120 user data 116 that corresponds to a current diet and calculate that reducing caloric intake to 1,800 may represent an average daily decrease of 200 calories for a user (if a user was consuming the 2,000 calorie standard daily intake), and increasing protein intake to 2 grams per kilogram body weight may increase a user's daily protein intake by 108 grams (if a user was a 90 kilogram individual and consuming the standard 0.8 gram protein per kilogram body weight daily protein value). In further non-limiting illustrative examples, a first machine-learning process 132 may reconcile opposing instructions by generating a different output that is a weekly meal plan, or provide a solution to reducing caloric intake by 200 calories per day, while increasing protein intake from 72 grams to 190 grams daily, such as suggesting instructions for a user to reduce carbohydrate intake by 30%, fat intake by 15%, and consume a protein powder supplement serving of 36 g protein, three times daily. A first machine-learning process 132 may accomplish this task by calculating the magnitude of effect of implementing each of the above instructions, as well as in combination, has toward achieving an objective of, for instance and without limitation ‘improving body composition’, at varying levels of improved body composition.

Continuing in reference to FIG. 1, identifying an instruction set to address an objective using a first machine-learning process 132 and may include generating an output of an instruction set to implementing a solution in addressing at least an objective. An output of an instruction set may include a solution, of a plurality of solutions, for addressing an objective and/or a plurality of objectives. A solution may include one instruction and/or a plurality of instructions. An instruction may contain a variety of additional data, including for instance and without limitation, an identifier that matches an instruction to an objective, and/or objectives, a calculated numerical value, variable, function, or the like, that describes an instruction's relative ability to address an objective, a tractability score for an objective based on the number of instructions directed to an objective, wherein a tractability score may be a numerical value, function, or the like, that describes the relative ability of a user to address and/or otherwise achieve an objective. An instruction may contain a variety of data that may include, for instance and without limitation, a signifier that matches an instruction to other instructions, such as a number for an instruction in a set of related instructions. A signifier may denote an alphanumerical code, number, value, or the like, describing a logical relationship between instructions, for instance a step, in series of steps, wherein all steps are fundamental to achieving a desired outcome.

Still referring to FIG. 1, computing device 104 may be configured to generate, using a second ranking process 144 and a first plurality of instructions, first ranked-ordered instruction set 108 for addressing a first rank-ordered goal set 124. A second ranking process 144 may be a ranking process that is the same as a first ranking process 128, as described above. Using a second ranking process 144 to combine a first ranked list of instructions for addressing at least an objective may include receiving a first rank-ordered instruction set 108 output by a first machine-learning process 132 stored and/or retrieved from a user database 120, as described above.

Continuing in reference to FIG. 1, computing device 104 may be configured for using a second ranking process 144 for generating the first rank-ordered instruction set 108 for addressing the rank-ordered goal set 124 which may include using a second ranking process 144 to weight each instruction using a ranking function, and/or weighting may be performed using any ranking algorithm and/or protocol suitable for performance of the first ranking process. In non-limiting illustrative embodiments, a ranking function may be a machine-learning model that is generated using training data retrieved from a user database 120, as described above, to train a machine-learning process. Alternatively or additionally, a ranking function may be a heuristic, function, vector, numerical table, matrix, or the like, that is retrieved as expert submission from an online repository, such as a research directory and/or scientific publication; a ranking function may be a model that is derived using training data that relates to other user outcomes from an instruction set. Weighting instructions using a ranking function may include applying a numerical value, factor, signifier, or the like to an instruction as it pertains to addressing an objective to determine a logical order for an instruction set. For instance, in non-limiting examples, weighting instructions may include using a ranking function that relates each instruction to its respective goal based on a scale of importance of each goal. In further non-limiting illustrative examples, weighting instructions in this manner may include using a ranking function that includes numerical values for how far a user is from attaining an objective, and weighting an instruction set using such a ranking function would place an instruction in a higher order in ranking in a set of instructions that are targeted towards an objective that is closer to completion; alternatively or additionally, if an objective is further from completion but is crucial to a user's immediate health, a ranking function may place instructions relating to that goal in a higher rank in a list of ranked instructions. Additionally, in non-limiting illustrative examples, weighting instructions may include ranking instructions based upon how tractable an objective is to user action, for instance and without limitation, a fitness goal compared to an educational goal, wherein a fitness goal of ‘improving cardiovascular endurance for running a 5 km race’ may be a highly tractable goal that is responsive to repetitive, short-term instructions such as 20 minutes of daily cardiovascular exercise, compared to an education goal of ‘getting on the Dean's list at university’, which is not as tractable of an objective and may require a more complicated and involved instruction set of short-term and long-term instructions.

Continuing in reference to FIG. 1, computing device 104 may be configured for using a second ranking process 144 for generating the first rank-ordered instruction set 108 for addressing the first rank-ordered goal set 124 which may include using a second ranking process 144 to determining a suitable timing for implementing each instruction. In non-limiting illustrative embodiments, a second ranking process 144 may determine suitable timing for instructions by using a ranking metric, score, or the like, that relates each instruction to how long it may take to complete, how crucial an objective is, how far away in time an objective may be for a user, or the like. For instance, in non-limiting illustrative examples, achieving a fitness goal of ‘improving cardiovascular endurance for running a 5 km race’ may include an instruction aimed at a specific amount of time of daily cardiovascular exercise, but the amount of running or when to being a fitness regimen to enact the instruction may depend on when the race is held. Such an objective may have been ranked by a first ranking process depending on the length of time until the race, and a ranking may reflect this information, likewise the information may be stored as part of an identifier in a database attached to the instruction. A first machine-learning process 132 may have identified and generated a series of instructions aimed at achieving said goal, including numerical data relating varying amounts, such as periods of time and running distances associated with cardiovascular exercise aimed at achieving the goal, and/or specific variations of the goal. For instance in non-limiting illustrative examples, ‘improving cardiovascular endurance for running a 5 km race in under X minutes,’ wherein X is a variable that may be determined by a user or retrieved from a ranking function or user database 120 for purposes of generating instructions. In non-limiting illustrative examples, a second ranking process 144 may adjust X up or down, may adjust frequency of the instruction containing X, and/or may increase or decrease the ranking of the instruction based on how far a user is from achieving an objective of X amount of time. A second ranking process 144 may use a ranking function to determine if an instruction is acute, concurrent to a second instruction, long-term, or the like, with respect to an instruction's optimal time of implementation within an instruction set. Alternatively or additionally, a signifier or other identifying element of data relating an instruction to its place in time among other instructions may be retrieved from a user database 120, or the like, and consist of one or more elements of data output by a first machine-learning process 132 identifying the instructions, as described above. A second ranking process 144 may then rank instructions based upon the suitable time frame for enacting an instruction.

Continuing in reference to FIG. 1, computing device 104 may be configured for using a second ranking process 144 for generating the first ranked-ordered instruction set 108 for addressing the rank-ordered goal set 124 may include generating a first rank-ordered instruction set 108 which combines the instructions from all steps, as described above.

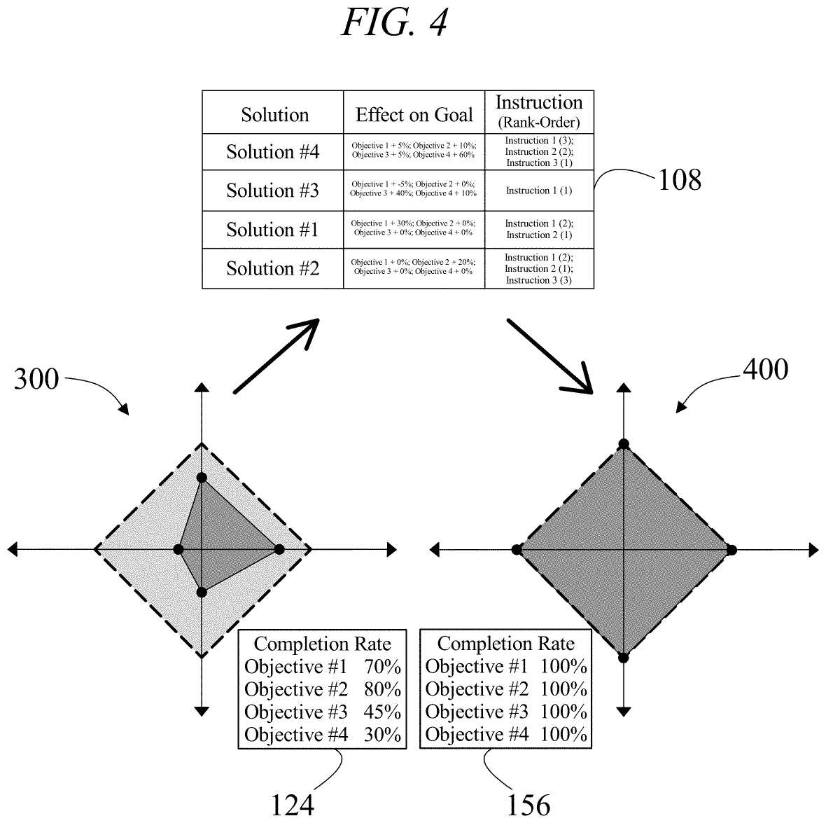

Referring now to FIG. 4, an exemplary embodiment of a first rank-ordered instruction set 108 after being applied 400 to a first rank-ordered goal set 124 is illustrated. A first rank-ordered goal set may be input into a first machine learning process 132 to identify and measure an effect of at least a solution towards achieving at least an objective. A second ranking process 144 may output a rank-ordered instruction set 108 as it relates to achieving a first rank-ordered goal set 124.

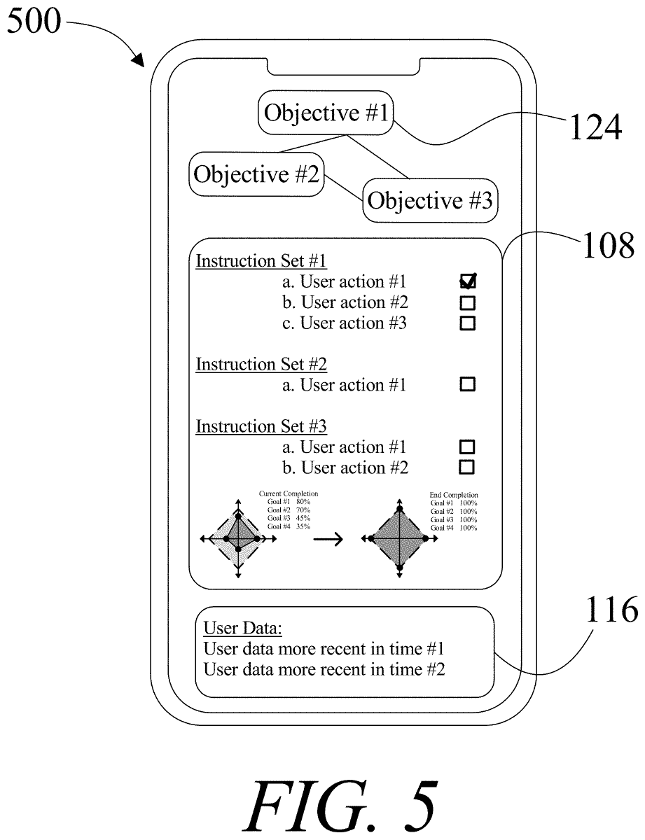

Referring now to FIG. 5, an exemplary embodiment of a user device 500 is illustrated. System 100 may provide a first rank-ordered instruction set 108 to a user device 500. User device 500 may communicate via a server, client device, or the like, as described in further detail below. A user device 500 may display a first rank-ordered instruction set 108 and a first rank-ordered goal set 124 that may be generated by a computing device 104. A user device 500 may display outputs via a graphical user interface, or by any other suitable method for displaying computer-generated outputs or numerical data, graphical data, text, or the like. Persons skilled in the art, upon reviewing the entirety of this disclosure, will be aware of various ways in which computer-generated outputs may be displayed via a user device to a user.

Continuing in reference to FIG. 5, user device 500 may be configured to receive a plurality of user data 116. A user device may prompt a user for user data 116 after an instruction set has been provided, in addition to any user data 116 that was collected to generate a first rank-ordered goal set 124. In non-limiting illustrative examples, user data 116 received after an instruction set has been provided may be a haptic and/or binary input, for instance tapping an instruction in the graphical user interface on a touch screen display to signify it has been completed, and/or indicating, clicking, or otherwise denoting in a designated ‘yes’ or ‘no’ input box in a user interface that an instruction has been completed by ‘checking off’ the instruction. Alternatively or additionally, user data 116 may be additional biological extraction data, such as without limitation, a blood test after adopting ketogenic diet instructions to achieve objectives of controlling blood sugar and preventing type-II diabetes. In non-limiting illustrative examples, such biological extraction user data 116 may be input by expert submission via a physician portal in a telemedicine platform to be stored and/or retrieved from a user database 120. In further non-limiting illustrative examples, user data 116 may be text input from a user via a user interface. Persons skilled in the art, upon reviewing the entirety of this disclosure, will be aware of various ways in which user interfaces may be used to collect, convert, and store user data 116 as described herein.

Continuing in reference to FIG. 5, computing device 104 may be configured to receive, from the user device 500, a plurality of user data 116, which may include user data 116 that is more recent in time than when a first instruction set was provided. Ranking process may determine, chronologically, how elements of user data 116 relate to rank-ordered goal sets and/or rank-ordered instruction sets. For instance and without limitation, a ranking process may recognize a chronological order from a timestamp signifier, identifier, alphanumerical code, or the like, that may be a value or datum attached to a user input, rank-ordered instruction set, and/or rank-ordered goal set.

Referring again to FIG. 1, computing device 104 may generate, using the plurality of user data 116, a second rank-ordered instruction set 148, wherein generating a second rank-ordered instruction set 148 using the plurality of user data 116 may include classifying user data 116 into categories, using a classification process 136 or any other suitable machine learning process, as user data 116 pertains to each instruction in a first set of instructions, as described above. User data 116 may be classified into subgroups based upon how user data 116 impacts an objective of a plurality of objectives. In non-limiting illustrative examples, meals logged by a user maybe be classified based on how the information from meal inputs relates to an objective of reducing body fat. In further non-limiting illustrative examples, meals logged by a user may be used to calculate daily caloric intake as related to at least an instruction that a user was provided for achieving an objective of reducing body fat. In non-limiting illustrative examples, the classification process 136 may assign user-reported meals to an objective of ‘reducing body fat,’ multiple objectives, or to no specific goal.

Continuing in reference to FIG. 1, computing device 104 may generate, using the plurality of user data 116, a second rank-ordered instruction set 148, wherein generating a second rank-ordered instruction set 148 using the plurality of user data 116 may include calculating, using a second machine learning process 152, the effect of at least a user action on a first set of objectives. A “user action,” as described herein is an element of user data 116 that directly relates to performing an instruction and/or working towards achieving an objective. A user action may be distinguished from other user data 116, such as biological extraction data, that may be used with a machine learning process or ranking process to determine an objective or generate an instruction set, but may not directly relate to performing an instruction that was provided to a user. User actions may be classified from user data 116 by a classification process 136 as the user action pertains to an instruction set for that goal, as described above.

A second machine-learning process 152 may accept categorized user actions as they relate to a set of instructions as an input and calculate the effect of at least an action on at least an instruction. A second machine-learning process 152 may be the same type of machine-learning process as a first machine-learning process 132, as described above. In non-limiting illustrative examples, user actions may be data logged via a wearable device as it pertains to a set of objectives. For instance, in non-limiting illustrative examples, a rank-ordered goal set for training a military pilot may be an objective derived from user biological extraction data that indicates a pilot should ‘limit G-force exposure to 6 G's’ and a user-specified goal to ‘run 10 km per week.’ User data 116 logged from a wearable device that tracks user movement, for instance and without limitation an accelerometer-gyrometer, may be categorized as it pertains to the two objectives. In further non-limiting illustrative examples, measurements of G-force may be collected and classified as sudden changes in velocity and/or force would be separated from accelerator-gyrometer data that describes athletic movements such as jogging, from low-impact movements, such as sitting and walking. The data can be separated and applied to the instruction sets using a classifier, which may be stored and/or retrieved from a user database 120. The categorized data, which includes a signifier matched to the corresponding goal and/or objectives, is then used as an input to a second machine-learning process 152. A second machine-learning process 152 may retrieve at least an instruction corresponding to at least an objective from a user database 120, and then perform a calculation to determine if an instruction was completed and/or to what degree an instruction was completed. A second machine-learning process 152 calculation may be any mathematical function such as subtraction, applying a ranking function, matrix, system of equations, or any other calculation performed by a machine-learning process, as described above. In further non-limiting illustrative examples, a second machine-learning process 152 may calculate from a military pilot's wearable accelerator-gyrometer data that throughout a week of training, no G-forces exceeded 5.6 G's, and the pilot ran a total of 12 km; a second machine-learning process may output a binary yes or no output after determining that a value of 6 G's was not exceeded, and may generate a numerical output that describes how user actions impacted a second goal, for instance, 120% of the weekly running total was met.

Continuing in reference to FIG. 1, computing device 104 may generate, second rank-ordered instruction set by determining, using a second machine learning process 152, a second plurality of objectives. Second plurality of objectives may reflect changes due to the more recent user data 116. A second machine-learning process 152 may determine how at least a user action from the more recent user data 116 impacts at least an objective by retrieving at least an instruction associated with that goal from a user database 120 and calculating if the instruction was met by the user action. A second machine-learning process 152 may calculate that an instruction was performed; likewise, a second machine-learning process 152 may calculate no effect, impact, or the like on an instruction. A second machine-learning process may calculate an effect and determine the effect on level of completion of an instruction on achieving an objective. An instruction's effect on achieving an objective may be a numerical value, function, matric, vector, of the like, that is a signifier, identifier, or the like attached as an element of datum to an instruction. A second machine-learning process 152 may evaluate the level of completion of each instruction as it applied to an objective and determine if an objective has been completed, or if it remains. Alternatively or additionally, more recent user data 116 may involve determining new objectives as it relates to achieving prior an objective or achieving an objective that have not been completed. A second machine learning process 152 may output a second plurality of objectives, wherein the second plurality of objectives reflects these changes due to the more recent user data 116.

Continuing in reference to FIG. 1, computing device 104 may determine a second rank-ordered goal set 156 using a third ranking process 160, wherein determining a second rank-ordered goal set 156 may include receiving a plurality of objectives updated using user data 116 output by a second machine learning process 152, and may include determining, using a ranking function, the relative importance of an objective. This may be implemented using any organization process and/or protocol, as described above. A third ranking process 160 may accept an input from a second machine-learning process 152 that is a second plurality of objectives updated to reflect changes in user data 116. A third ranking process 160 may use a similar objective function as a first ranking process 128 and/or a second ranking process 144, as described above. A ranking process may include any of the functions described above, such as a linear objective function that may input a plurality of user-reported objectives, and rank the objectives by a variety of factors, for instance without limitation, by impact to health, and output a rank-ordered list of objectives ranked by that function. Determining a second rank-ordered goal set 156 may include using a third ranking process 160 using a ranking function, metric, or the like, to determine the relative importance, timing, positioning, or the like, of an objective, as described above. In non-limiting illustrative embodiments, an objective function may use a ranking function to determine a rank-order for objectives based on a numerical value, index, matrix, or the like, to determine the goal rank order for a user. In non-limiting illustrative examples ranking function may be at least a value that was retrieved from a user database 120, calculated by a machine-learning process, or otherwise obtained that provides qualitative and/or quantitative guidance in determining a rank for an objective. Ranking function may contain values derived from user data 116 to determine a priority listing for objectives based on, for instance without limitation severity of health concern.

Continuing in reference to FIG. 1, computing device 104 may identify a second plurality of instructions, wherein identifying an instruction set using a third machine learning process 164 may include receiving a second rank-ordered goal set 156 from a third ranking process 160. A third machine-learning process 164 may include a machine-learning process as described above, including without limitation any machine-learning process suitable for generating first rank-ordered goal set. Third machine-learning process 164 may be the same as a first machine-learning process 132 and/or a second machine-learning process 152, as described above. Third machine-learning process 164 may receive a second rank-ordered goal set 156 from a third ranking process 160, for instance as updated from more recent user data 116 as described above.

Continuing in reference to FIG. 1, computing device 104 may identify second plurality of instructions using third machine learning process 164 by retrieving at least a solution for addressing an objective from a user database 120, using a third machine learning process 164. In non-limiting illustrative embodiments, a third machine-learning process 164 may determine a course of action for a user to work towards achieving at least an objective by retrieving information, for instance from a user database 120, as described above. In further non-limiting embodiments, a third machine learning process 164 retrieving information from a user database 120 may query, for instance and without limitation, solutions for achieving an objective related to improving a credit score, calculating the anticipated impact of each solution on the second rank-ordered list of goal, and then filter solutions based on a variety of methods, as described herein.

Continuing in reference to FIG. 1, computing device 104 identifying second plurality of instructions using third machine learning process 164 may include measuring an effect of a solution on an objective. Third machine-learning process 164 may measure the effect of an instruction, describing an incremental step of a solution, on an objective of a second plurality of objectives, by factoring completion of an instruction towards achieving an objective, as described above. Measuring an effect of an instruction may include performing any mathematical function such as subtraction, applying a ranking function, matrix, system of equations, or any other calculation performed by a machine-learning process. In non-limiting illustrative embodiments, a third machine-learning process may retrieve at least an element of data from a user database 120, such as a metric, indicator, or the like, that relates level of completion of an instruction towards achieving an objective, as described above.

Continuing in reference to FIG. 1, computing device 104 identifying second plurality of instructions using third machine learning process 164 may include eliminating redundant solutions in addressing at least an objective, of a plurality of a second rank-ordered goal set 156, as described above.

Continuing in reference to FIG. 1, computing device 104 identifying a second plurality of instructions using third machine learning process 164 may include reconciling opposing solutions in addressing at least an objective. For instance and without limitation, a third machine-learning process 164 may reconcile solutions that may be opposing in practice in addressing at least an objective, as described above.

Continuing in reference to FIG. 1, computing device 104 identifying a second plurality of instructions using a third machine learning process 164 may include generating an output of an instruction set to implementing a solution in addressing at least an objective. An output of an instruction set may include a solution, of a plurality of solutions, for addressing an objective and/or a plurality of objectives. A solution may include one instruction and/or a plurality of instructions. An instruction may contain a variety of data, including for instance and without limitation, an identifier that matches an instruction to an objective, and/or objectives, a calculated numerical value, variable, function, or the like, that describes an instruction's relative ability to address an objective, a tractability score for an objective based on the number of instructions directed to an objective, wherein a tractability score may be a numerical value, function, or the like, that describes the relative ability of a user to address and/or otherwise achieve an objective, as described above. A tractability score for a second plurality of objectives may be updated by training a machine-learning process 164 using training data 140, wherein training data 140 may contain user data 116 that is more recent than a first instruction set, as described above. An instruction may contain a variety of data that may include, for instance and without limitation, a signifier that matches an instruction to other instructions, such as a number for an instruction in a set of related instructions. A signifier may denote an alphanumerical code, number, value, or the like, describing a logical relationship between instructions, for instance a step, in series of steps, wherein all steps are fundamental to achieving a desired outcome.

Continuing in reference to FIG. 1, computing device 104 generating a second rank-ordered instruction set 148 using a fourth ranking process 168 may include receiving, a second set of instructions output by a third machine learning process 164. A fourth ranking process 168 may be a ranking process that is the same as a first ranking process 128, as described above. Using a fourth ranking process 168 to combine and output a second rank-ordered instruction set 148 for addressing at least an objective or a second rank-ordered goal set 156 may include receiving a second set of instructions output by a third machine-learning process 164 stored and/or retrieved from a user database 120, as described above.

Continuing in reference to FIG. 1, computing device 104 generating a second rank-ordered instruction set 148 using a fourth ranking process 168 may include weighing each instruction using a ranking function. In non-limiting illustrative embodiments, a ranking function may be a machine-learning model that is generated using training data 140 retrieved from a user database 120, as described above, to train a machine-learning process. Alternatively or additionally, a ranking function may be a heuristic, function, vector, numerical table, matrix, or the like, that is retrieved as expert submission from an online repository, such as a research directory and/or scientific publication; a ranking function may be a model that is derived using training data that relates to other user outcomes from an instruction set. Weighting instructions using a ranking function may include applying a numerical value, factor, signifier, or the like to an instruction as it pertains to addressing an objective to determine a logical order for an instruction set, as described above.

Continuing in reference to FIG. 1, computing device 104 may generate a second rank-ordered instruction set 148 using a fourth ranking process 168, by determining the suitable timing for implementing each instruction. In non-limiting illustrative embodiments, a fourth ranking process 168 may determine suitable timing for instructions by using a ranking function that relates each instruction to how long it may take to complete, how crucial an objective is, how far away in time an objective may be for a user, or the like, as described above. A third machine-learning process 164 may have identified and generated a series of instructions aimed at achieving an objective, including numerical data relating varying amounts, such as periods of time, magnitude of action, and the like, aimed at achieving the goal, and/or specific variations of the goal, as described above. A fourth ranking process 168 may use a ranking function to determine if an instruction is acute, concurrent to a second instruction, long-term, or the like, with respect to an instruction's optimal time of implementation within an instruction set, as described above. Alternatively or additionally, a signifier or other identifying element of data relating an instruction to its place in time among other instructions may be stored and/or retrieved from a user database 120, or the like, and consist of one or more elements of data output by a third machine-learning process 164 identifying the instructions, as described above. A fourth ranking process 168 may then rank instructions based upon the suitable time frame for enacting an instruction.

Continuing in reference to FIG. 1, computing device 104 may generate a second rank-ordered instruction set 148 using a fourth ranking process 168 by generating a second rank-ordered list which combines the instructions, as described above.

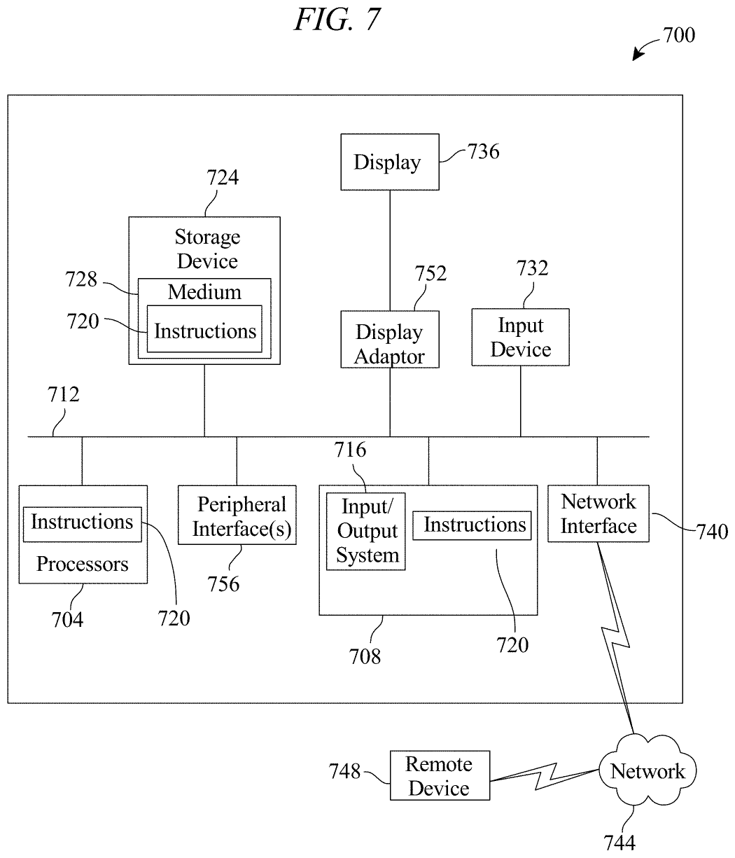

Referring now to FIG. 7, an exemplary embodiment of a method 700 of generating rank-ordered instruction sets using a ranking process, is illustrated. At step 705, computing device 104 may be configured for generating a first rank-ordered list of instructions.

At step 710, computing device 104 may be configured for receiving a plurality of user objectives. Receiving a plurality of objectives may include receiving at least a user-reported goal. Receiving a plurality of objectives may include receiving at least an objective determined from a plurality of user-reported data.

At step 715, computing device 104 may be configured for determining, using a first ranking process and a plurality of objectives, a rank-ordered goal set. Determining a rank-ordered goal set further comprises using a ranking process using a ranking function to determine the relative importance of an objective.

At step 720, computing device 104 may be configured for identifying, using a first machine learning process and ranked-ordered goal set, an instruction set including a plurality of instructions, wherein the plurality of instructions includes an instruction for addressing each goal of the plurality of objectives. Identifying an instruction set to address an objective may include receiving a plurality of rank-ordered objectives from a first ranking process. Identifying an instruction set to address an objective may include measuring, using the machine learning process an effect of a solution on an objective. Identifying an instruction set to address an objective may include eliminating, using the machine learning process, redundant solutions in addressing at least an objective. Identifying an instruction set to address an objective may include reconciling, using the machine learning process, opposing solutions in addressing at least an objective. Identifying an instruction set to address an objective may include generating an output of an instruction set to implementing a solution in addressing at least an objective.

At step 725, computing device 104 may be configured for generating, using a second ranking process and a first plurality of instructions, a first rank-ordered list of instructions for addressing the rank-ordered goal set. Using a second ranking process to generate a first ranked list of instructions for addressing at least an objective may include receiving, a first set of instructions output by a first machine learning process. Generating, using a second ranking process and a first plurality of instructions, a first rank-ordered list of instructions for addressing the rank-ordered goal set may include weighing each instruction using a ranking function. Generating, using a second ranking process and a first plurality of instructions, a first rank-ordered list of instructions for addressing the rank-ordered goal set may include determining the suitable timing for implementing each instruction. Generating, using a second ranking process and a first plurality of instructions, a first rank-ordered list of instructions for addressing the rank-ordered goal set may include generating a second rank-ordered list which combines the instructions.

At step 730, computing device 104 may be configured for providing the rank-ordered goal set to a user device. Computing device 104 may be configured to provide data to a user device, as described above.

At step 735, computing device 104 may be configured for receiving, from the user device, a plurality of user data 116. Receiving from the user device, a plurality of user data 116 may include providing at least a first instruction set to a user. Receiving from the user device a plurality of user data 116 may include receiving user data 116 more recent in time than when a first instruction set was provided.

At step 740, computing device 104 may be configured for generating, using the plurality of user data 116, a second rank-ordered list of instructions. Generating a second rank-ordered list of instructions using the plurality of user data 116 may include classifying, using a classification process, user data 116 into categories as user data 116 pertains to each instruction in a first set of instruction. Generating a second rank-ordered list of instructions using the plurality of user data 116 may include calculating, using a second machine learning process, the effect of at least a user action on a first set of objectives. Generating a second rank-ordered list of instructions using the plurality of user data 116 may include determining, using a second machine learning process, a second plurality of objectives, wherein a second plurality of objectives reflects changes due to the more recent user data 116. Determining a second rank-ordered goal set using a third ranking process may include receiving a plurality of objectives updated using user data 116 output by a second machine learning process. Determining a second rank-ordered goal set using a third ranking process may include determining, using a ranking function, to determine the relative importance of an objective.

Generating a second rank-ordered list of instructions using the plurality of user data 116 may include identifying an instruction set using a third machine learning process. Identifying an instruction set using a third machine learning process may include receiving a second plurality of rank-ordered objectives from a third ranking process. Identifying an instruction set using a third machine learning process may include retrieving from a database, using a third machine learning process, at least a solution for addressing an objective. Identifying an instruction set using a third machine learning process may include measuring an effect of a solution on an objective. Identifying an instruction set using a third machine learning process may include eliminating redundant solutions in addressing at least an objective. Identifying an instruction set using a third machine learning process may include reconciling opposing solutions in addressing at least an objective. Identifying an instruction set using a third machine learning process may include generating an output of an instruction set to implementing a solution in addressing at least an objective.

Generating a second rank-ordered instruction set using a fourth ranking process may include receiving, a second set of instructions output by a second machine learning process. Generating a second rank-ordered instruction set using a fourth ranking process may include weighing each instruction using a ranking function. Generating a second rank-ordered instruction set using a fourth ranking process may include determining the suitable timing for implementing each instruction. Generating a second rank-ordered instruction set using a fourth ranking process may include generating a second rank-ordered list which combines the instructions.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A system for generating a rank-ordered instruction set using a ranking process, the system comprising at least a computing device, wherein the at least a computing device comprises a processor, and wherein the at least a computing device is configured to:
generate a first rank-ordered list of instructions, wherein generating further comprises:
receiving a plurality of user objectives;
determining, using a first ranking process and the plurality of objectives, a rank-ordered objective set;
generating, using a first machine learning process and the rank-ordered objective set, a first instruction set including a plurality of instructions as a function of a biological extraction, wherein the biological extraction includes a physically extracted datum of a user, and wherein the plurality of instructions includes an instruction for addressing each objective of the plurality of user objectives, and wherein the first machine learning process is trained as a function of a first training set that correlates the biological extraction and the rank-ordered objective set; and
generating, using a second ranking process and the plurality of instructions, the first rank-ordered list of instructions for addressing the rank-ordered objective set;
provide the first rank-ordered list of instructions to a user device;
receive, from the user device, a plurality of user data; and
generate, using the plurality of user data, a second rank-ordered list of instructions,
wherein generating the second rank-ordered list of instructions further comprises:
calculating, using a second machine learning process, the effect of at least a user action on the plurality of user objectives, and wherein the user action includes information on the user's implementation of an instruction, from the first rank-ordered list of instructions, to achieve a human health objective of the user from the plurality of user objectives.

2. The system of claim 1, wherein receiving a plurality of user objectives further comprises:
receiving at least an objective determined from a plurality of user-reported data.

3. The system of claim 1, wherein determining the rank-ordered objective set using the first ranking process further comprises using a ranking function to determine relative importance of each objective of the plurality of user objectives and determine the rank-ordered objective set as a function of the relative importance of each objective of the plurality of user objectives.

4. The system of claim 1, wherein generating the first instruction set to address an objective further comprises:
retrieving from a database, using the first machine-learning process, at least an instruction corresponding to an objective; and
measuring, using the first machine-learning process, an effect of a solution on the objective.

5. The system of claim 1, wherein generating the first rank-ordered list of instructions further comprises:
weighting each instruction of the first instruction set using a ranking function that relates to a relative importance of an objective;
determining a suitable timing for implementing each instruction; and
generating the first rank-ordered list of instructions as a function of the first weighted instruction set and the suitable timing.

6. The system of claim 1, wherein:
the first rank-ordered list of instructions has a time of production;
the plurality of user data has a time of reception; and
the time of reception is later than the time of production.

7. The system of claim 1, wherein the computing device is configured to generate the second rank-ordered list of instructions by:
classifying, using a classification process, the plurality of user data into categories pertaining to instructions in the first rank-ordered list of instructions;
calculating, using the second machine-learning process, the effect of the at least a user action on the plurality of user objectives corresponding to the plurality of classified user data; and
determining, as a function of the second machine-learning process and the plurality of classified user data, a second plurality of objectives.

8. The system of claim 7, wherein the system is further configured to generate a second rank-ordered objective set using a third ranking process.

9. The system of claim 8, wherein generating the second rank-ordered list of instructions further comprises generating, as a function of a third machine-learning process and the second rank-ordered objective set, a second instruction set.

10. The system of claim 9, wherein generating the second rank-ordered list of instructions further comprises:
weighting each instruction of the second instruction set using a ranking function that at least relates to a relative importance of an objective;
determining a suitable timing for implementing each instruction; and
generating the second rank-ordered list of instructions as a function of the second weighted instruction set and the suitable timing.

11. A method for generating a rank-ordered instruction set using a ranking process implemented by a system comprising at least a computing device, wherein the at least a computing device comprises a processor, and wherein the at least a computing device is configured to:
generate a first rank-ordered list of instructions, wherein generating further comprises:
receiving a plurality of user objectives;
determining, using a first ranking process and the plurality of user objectives, a rank-ordered objective set;
identifying, as a function of a first machine-learning process and the rank-ordered objective set, a first instruction set including a plurality of instructions as a function of a biological extraction, wherein the biological extraction includes physically extracted datum of a user, and wherein the plurality of instructions includes an instruction for addressing each objective of the plurality of user objectives, and wherein the first machine learning process is trained as a function of a first training set that correlates the biological extraction and the rank-ordered objective set; and
generating, using a second ranking process and the first plurality of instructions, the first rank-ordered list of instructions for addressing the rank-ordered objective set;
provide the first rank-ordered list of instructions to a user device;
receive, from the user device, a plurality of user data; and
generate, using the plurality of user data, a second rank-ordered list of instructions,
wherein generating the second rank-ordered list of instructions further comprises:
calculating, using a second machine learning process, the effect of at least a user action on the plurality of user objectives, and wherein the user action includes information on the user's implementation of an instruction, from the first rank-ordered list of instructions, to achieve a human health objective of the user from the plurality of user objectives.

12. The method of claim 11, wherein receiving a plurality of user objectives further comprises:
receiving at least an objective determined from a plurality of user-reported data.

13. The method of claim 11, wherein determining the rank-ordered objective set using the first ranking process further comprises using a ranking function to determine relative importance of each objective of the plurality of user objectives and determine the rank-ordered objective set as a function of the relative importance of each objective of the plurality of user objectives.

14. The method of claim 11, wherein identifying the first instruction set further comprises:
retrieving from a database, using the first machine-learning process, at least an instruction corresponding to an objective; and
measuring, using the first machine-learning process an effect of a solution on the objective.

15. The method of claim 11, wherein generating the first rank-ordered list of instructions further comprises:
weighting each instruction of the first instruction set using a ranking function that relates to a relative importance of an objective;
determining a suitable timing for implementing each instruction; and
generating the first rank-ordered list of instructions as a function of the first weighted instruction set and the suitable timing.

16. The method of claim 11, wherein:
The first rank-ordered list of instructions has a time of production;
the plurality of user data has a time of reception; and
the time of reception is later than the time of production.

17. The method of claim 11, wherein the computing device is configured to generate the second rank-ordered list of instructions by:
classifying, using a classification process, the plurality of user data into categories pertaining to instructions in the first rank-ordered list of instructions;
calculating, using the second machine-learning process, the effect of the at least a user action on the plurality of user objectives corresponding to the plurality of classified user data; and
determining, as a function of the second machine-learning process and the plurality of classified user data, a second plurality of objectives.

18. The method of claim 17, wherein the method further comprises generating a second rank-ordered objective set using a third ranking process.

19. The method of claim 18, wherein generating the second rank-ordered list of instructions further comprises generating, as a function of a third machine-learning process and the second rank-ordered objective set, a second instruction set.

20. The method of claim 19, wherein generating the second rank-ordered list of instructions further comprises:
weighting each instruction of the second instruction set using a ranking function that at least relates to a relative importance of an objective;
determining a suitable timing for implementing each instruction; and
generating the second rank-ordered list of instructions as a function of the second weighted instruction set and the suitable timing.
